(19) United States
(12) Patent Application Publication
(10) Pub. No.: US 2011/0112579 A1
(43) Pub. Date: May 12, 2011

(54) ROD AND METHOD OF INSERTION

(76) Inventors: DECLAN PATRICK BRAZIL, Cabarita (AU); Robert David Labrom, Taringa (AU)

(21) Appl. No.: 12/913,507

(22) Filed: Oct. 27, 2010

Related U.S. Application Data
(60) Provisional application No. 61/255,799, filed on Oct. 28, 2009.

(51) Int. Cl. A61B 17/70; A61B 17/88

(52) U.S. Cl. 606/264; 606/246; 606/279

(57) ABSTRACT

A spinal fusion system has at least one spinal rod including a longitudinal member having at least one hollow extending therethrough, and at least one rod fenestration that extends through the longitudinal member. There are at least two bone screws for attachment to a spine. Each bone screw includes a threaded shaft for location within the spine, and a head attached to the threaded shaft. The head is able to be used for attachment to a rod, and the head includes at least one fenestration. At least one spinal rod is attached to the respective heads of the bone screws.

### FIELD OF THE INVENTION

This invention relates to a rod and method of insertion. In particular, the invention relates to a rod used for spinal fusion and therefore will be described in this context. However, it should be appreciated that the rod may be used for other surgical procedures, such as any fixation of two bone segments.

### BACKGROUND OF THE INVENTION

Posterior spinal fusion has been well understood over the decades and remains a common surgical procedure that is required to treat spinal pathologies, such as degenerative pathologies, deformity, tumours and traumatic fractures. The combination of metallic bone screws and associated rods has also been well described and adds to the biomechanical stability of the spinal level or levels being fused. Further, the use of metallic bone screws and associated rods adds to an increase in fusion rates. However, the addition of bone graft to the posterior spinal bone surfaces to achieve successful biological spinal fusion remains one of the key components to the surgical technique of posterior spinal fusion.

Advances in minimally invasive surgical (MIS) techniques has improved patient morbidity outcomes, yet offers new challenges to surgeons to expose and to deliver bone graft to posterior bone surfaces of the spinal column, especially the transverse processes. Traditionally, bone graft of various types is added to the space between the transverse processes between two vertebrae that are being fused. This postero-lateral gutter space between the transverse processes can be difficult to reach and requires a degree of muscle stripping to expose this space. Percutaneous bone screw placement has also been popularised, yet offers a new challenge for the adequate exposure of the postero-lateral gutter for the application and delivery of bone graft materials.

It is an object of the invention to overcome and/or alleviate one or more of the above disadvantages or provide the consumer with a useful or commercial choice.

### SUMMARY OF THE INVENTION

In one form, although not necessarily the only or broadest form, the invention resides in a spinal rod able to be used with any bone fixation device, the spinal rod comprising:

a longitudinal member having at least one hollow extending therethrough.

The longitudinal member is typically cylindrical in shape. The diameter of the longitudinal member may be the same or similar to the dimension of spinal rods currently on the market. However, it is envisaged that the longitudinal member may be of any suitable diameter and/or length.

The longitudinal member may be made from a combination of segments. Each segment may be solid, hollow, or a combination thereof.

The hollow may extend completely or partially through the longitudinal member.

Preferably, the spinal rod comprises at least one fenestration. Normally there are a plurality of rod fenestrations that extend through the longitudinal member.

Preferably, the longitudinal member is made from a material or materials that allow bone growth on the surface of the longitudinal member. More preferably, the longitudinal member will allow bone growth through the rod fenestrations and within the hollow of the longitudinal member. The material used may be titanium alloy, CrCo, 316L stainless steel, PEEK™, Nitinol, ceramic and any other polymer, metal or any material. The longitudinal member may be treated with a roughening process, such as blasting or hydroxyl-apatite coating, as to assist in permitting bone on-growth.

The longitudinal member may be straight, pre-bent to a set arc or able to be bent at the time of the surgery.

The longitudinal member may have an internal design structure that adds strength in a certain direction or plane. For example, the longitudinal member may include a ridged beam or strengthening ribs.

It is preferable that the hollow of the longitudinal member is sized to permit the easy passage of bone graft material along the length of the longitudinal member. The internal diameter dimensions of the hollow may permit programmed bone graft flow. That is, the internal dimensions of the hollow member may or may not be uniform. It will be designed so as to optimise flow dynamics for the graft material. The hollow may permit passage of graft material to adjacent bony structures to encourage posterior spinal bone grafting between two spinal levels.

The hollow of the longitudinal member may be pre-packed with graft material.

The end of the hollow may be capped to act as a graft restrictor. Alternatively, the hollow may be open at both ends, closed at both ends or a combination thereof.

The rod fenestrations may be of any suitable size and/or shape. The rod fenestrations typically are in fluid communication with the hollow. The rod fenestrations can be arranged posteriorly, anteriorly, medially and/or laterally

The rod fenestrations may be preferentially placed to direct graft material into the posterior spinal lateral gutter and/or medially placed facet joint area or any area above/below or either side of the longitudinal member.

The hollow and/or rod fenestrations could include a design feature or be made of a material that permits a particular flow of graft and/or fluid in a certain direction and/or speed. That is, the hollow and/or rod fenestrations may include channels, ridges, barriers or similar. A delivery port may be located on the longitudinal member.

The delivery port may double as a rod fenestration. The delivery port may be located on any portion of the longitudinal member. Preferably, the delivery port is located at one end of the longitudinal member.

A delivery tool for inserting graft material may be connected to the delivery port. The delivery tool may be hand driven, hydraulic, motorized or pneumatic.

The hollow and fenestrated rods could include a design feature or be made of a material that permits a dynamic or a movable feature. A motion couple to a solid rod section could be included. A helical shape to a segment of the rod or a material choice, such as a memory metal alloy, could be included to permit some motion to permit a dynamic fusion or stabilization construct.

In another form, the invention resides in a bone screw comprising:

a threaded shaft for location to a spine;

a screw head attached to the threaded shaft, the head able to be used for attachment to a rod;

wherein the screw head includes at least one fenestration.

The shape and size could be variable to regulate rate and direction of flow.

The screw head fenestration could be on one side only or both sides of the screw head. The screw head could be treated to permit on-growth of bone. For example, the screw head could be acid treated to permit on-growth, roughened or could be hydroxy-apatite coated.

The screw head may be made of titanium alloy, 316L stainless steel, chrome cobalt, Nitinol or any other suitable material.

The screw head could be fixed or mobile to the shaft.

In another form, the invention resides in a spinal fusion system comprising:

at least one spinal rod comprising a longitudinal member having at least one hollow extending through the longitudinal member; and a plurality of rod fenestrations that extend through the longitudinal member; and

at least two bone screws for attachment to a spine, each bone screw comprising a threaded shaft for location within the spine, a head attached to the threaded shaft, the head able to be used for attachment to a rod, the head including at least one fenestration;

wherein at least one spinal rod is attached to the respective heads to the bone screws.

Preferably, the rod fenestrations are aligned with screw head fenestrations to allow graft material to pass from the longitudinal member and through the screw head.

In another form, the invention resides in a method of surgery including the steps of:

attaching a spinal rod to at least one bone screw, the spinal rod comprising a longitudinal member having at least one hollow extending through the longitudinal member; and a plurality of rod fenestrations that extend through the longitudinal member; and

injecting bone graft into the hollow of the longitudinal member.

The method may further include one or more of the follow steps including:

setting the bone screw in a spine, each bone screw comprising a threaded shaft for location within the spine, a head attached to the threaded shaft, the head able to be used for attachment to a rod, the head including at least one fenestration; wherein the shaft is rotated so that the fenestration in the head is in a desired location;

rotating the rod with respect to the bone screw such that a rod fenestration is located adjacent a screw head fenestration.

### BRIEF DESCRIPTION OF THE DRAWINGS

Embodiments of the invention will be described with reference to the accompanying drawings in which:

FIG. 1A is an end view of a spinal rod according to a first embodiment of the invention;

FIG. 1B is a perspective view of a spinal rod according to FIG. 1A;

FIG. 2A is an end view of a spinal rod according to a second embodiment of the invention;

FIG. 2B is a perspective view of a spinal rod according to FIG. 2A;

FIG. 3A is a perspective view of a spinal rod according to a third embodiment of the invention;

FIG. 3B is a side sectional view of a spinal rod according to FIG. 3A;

FIG. 4A is a perspective view of a spinal rod according to a fourth embodiment of the invention;

FIG. 4B is a side sectional view of a spinal rod according to FIG. 4A;

FIG. 5A is a perspective view of a spinal rod according to a fifth embodiment of the invention;

FIG. 5B is a side sectional view of a spinal rod according to FIG. 5A;

FIG. 6A is a perspective view of a spinal rod according to a sixth embodiment of the invention;

FIG. 6B is a side sectional view of the spinal rod of FIG. 6A attached to two bone screws;

FIG. 7A is a perspective view of a spinal rod according to a seventh embodiment of the invention;

FIG. 7B is a side sectional view of the spinal rod of FIG. 7A attached to two bone screws;

FIG. 8A is a perspective view of a spinal rod according to a eighth embodiment of the invention;

FIG. 8B is a perspective view of the spinal rod of FIG. 8A attached to two bone screws with graft material located within the spinal rod;

FIG. 9A is a perspective view of a spinal rod according to a ninth embodiment of the invention;

FIG. 9B is a perspective view of the spinal rod of FIG. 9A attached to two bone screws with graft material located within the spinal rod;

FIG. 10A is a perspective view of a spinal rod according to a tenth embodiment of the invention;

FIG. 10B is a perspective view of the spinal rod of FIG. 10A attached to two bone screws with graft material located within the spinal rod;

FIG. 11A is a perspective view of a spinal rod according to a eleventh embodiment of the invention;

FIG. 11B is a perspective view of the spinal rod of FIG. 11A attached to two bone screws with graft material located within the spinal rod;

FIG. 12 is a perspective view of a spinal rod attached to two bone screws with graft material being introduced through a pipe;

FIG. 13 is a perspective view of a spinal rod attached to two bone screws with graft material being introduced through a locking cap;

FIG. 14 is a perspective view of a spinal rod attached to two bone screws with graft material being introduced through a pipe;

FIG. 15A is a perspective view of a delivery tool according to an embodiment of the invention;

FIG. 15B is a perspective view of the delivery tool of FIG. 15A in use in the delivery graft material in the hollow of a spinal rod;

FIG. 16A is a perspective view of a further delivery tool adjacent an associated spinal rod according to an embodiment of the invention; and

FIG. 16B is a perspective view of the delivery tool of FIG. 16A in use in the delivery graft material in the hollow of a spinal rod shown in FIG. 16A.

### DETAILED DESCRIPTION OF THE PREFERRED EMBODIMENT

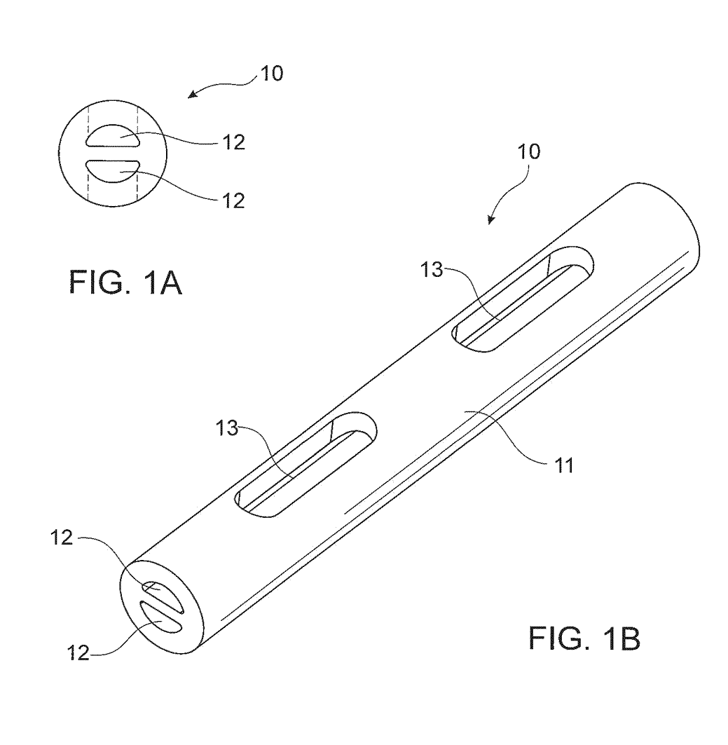

FIGS. 1A and 1B shows a perspective view of a spinal rod 10 for use in spinal surgery, especially with respect to spinal fusion. The spinal rod 10 is formed from a longitudinal member 11 made of titanium. However, it should be appreciated that other materials may be used. Two hollows 12 extend through the length of the longitudinal member 11. The two hollows 12 are semi-circular in transverse cross section.

Four rod fenestrations 13 extend through the longitudinal member 11. The rod fenestrations 13 are elliptical in shape. However, it should be appreciated that the shape and size of the rod fenestrations 13 may be varied according to design requirements. Two rod fenestrations 13 are located on one side of the longitudinal member 11 with the other two rod fenestrations 13 being located on the opposite side of the longitudinal member 11. Two rod fenestrations 13 are fluidly connected to one of the two hollows 12 whilst the other two rod fenestrations 13 are fluidly connected to the other of the two hollows 12.

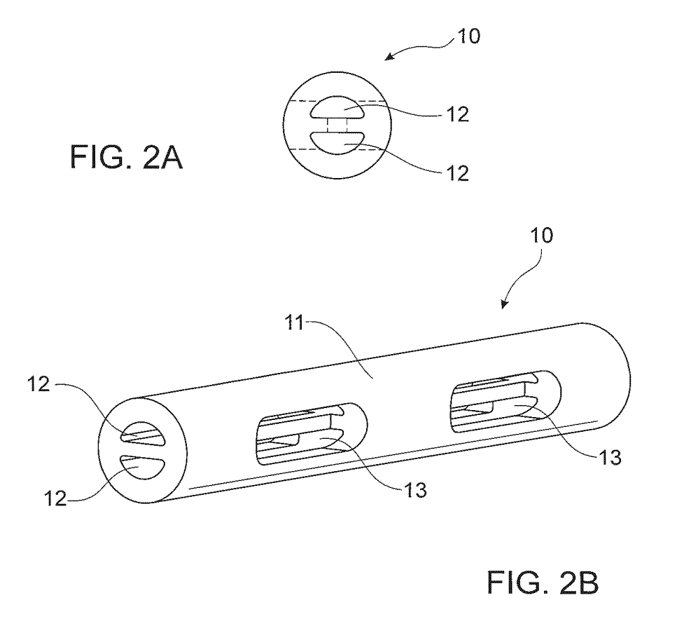

FIGS. 2A and 2B show a second embodiment of a spinal rod 10. In this embodiment, there are again two rod fenestrations 13. However, each of these rod fenestrations 13 are fluidly connected to the two hollows 12.

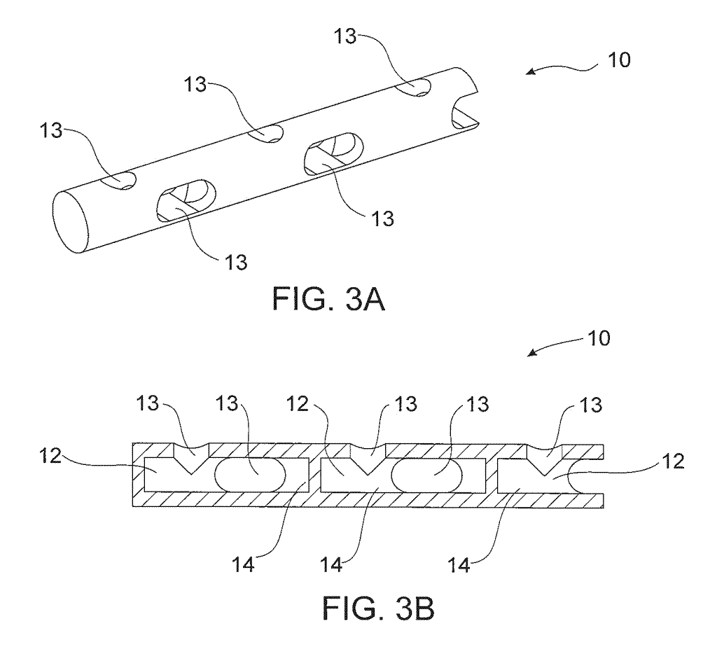

FIGS. 3A and 3B show a third embodiment of a spinal rod 10. In this embodiment, the longitudinal member 11 has three individual compartments 14. Each of these compartments 14 has a hollow 12, a circular rod fenestration 13 and an elliptical rod fenestration 13. One of the compartments 14 has an open end whilst the other compartments are enclosed.

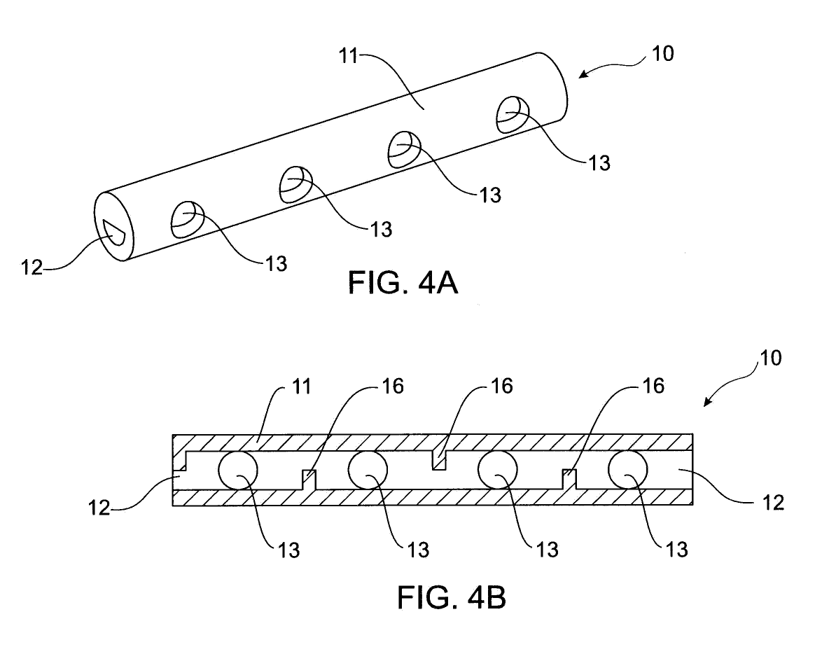

FIGS. 4A and 4B show a fourth embodiment of a spinal rod 10. In this embodiment, there are four circular rod fenestrations 13. Flow restrictors 16 are located within the single hollow 12. One end of the hollow 12 is also semi-circular in transverse cross-section to vary flow.

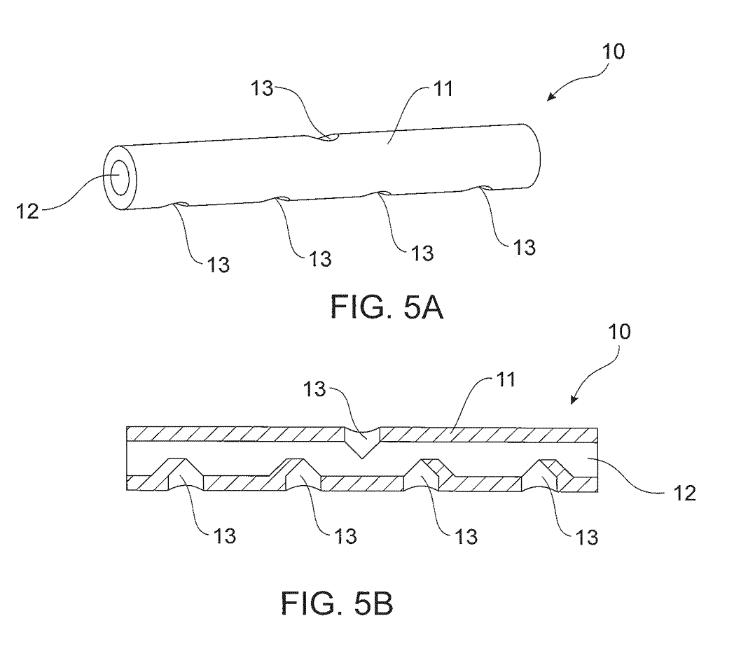

FIGS. 5A and 5B show a fifth embodiment of a spinal rod 10. In this embodiment, there is a single hollow 12 and five rod fenestrations 13. Four rod fenestrations 13 are located on one side of the longitudinal member 11 with the other rod fenestration 13 diametrically opposed. A flow control device 17, in the form of a barb, is located adjacent each of the four rod fenestrations 13. Each of the barbs face toward the fifth rod fenestration 13.

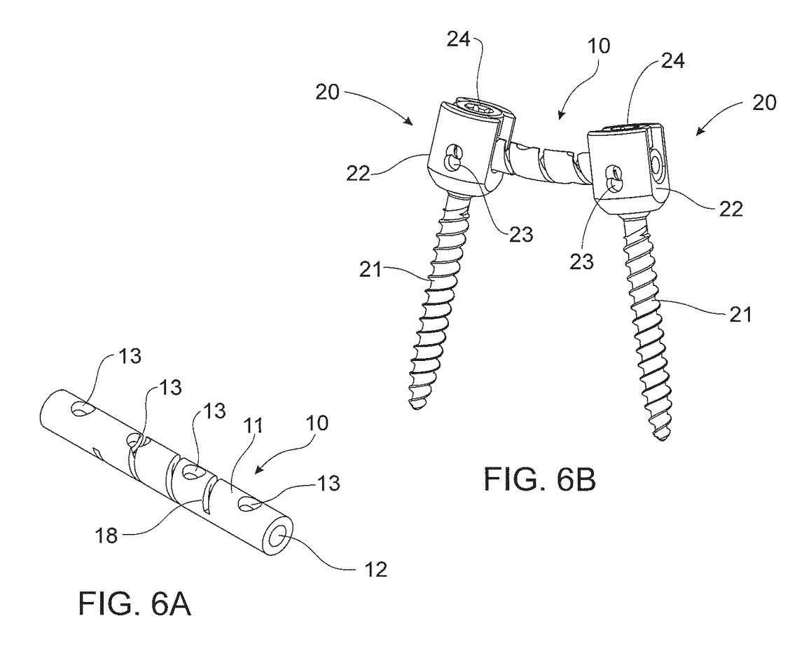

FIG. 6A shows a sixth embodiment of a spinal rod 10. The spinal rod 10 again has a hollow 12 extending through the longitudinal member 11 with four rod fenestrations 13 extending through the longitudinal member 11. A helical channel 18 extends through the centre of the longitudinal member 11 to make for easier bending of the longitudinal member 11.

FIG. 6B shows the spinal rod 10, shown in FIG. 6A, attached to two bone screws 20. Each bone screw 20 includes a threaded shaft 21 and a screw head 22. Two screw head fenestrations 23 are located on each screw head 22. The spinal rod 10 is attached to the screw head 22 using locking caps 24 as is standard practice in the art.

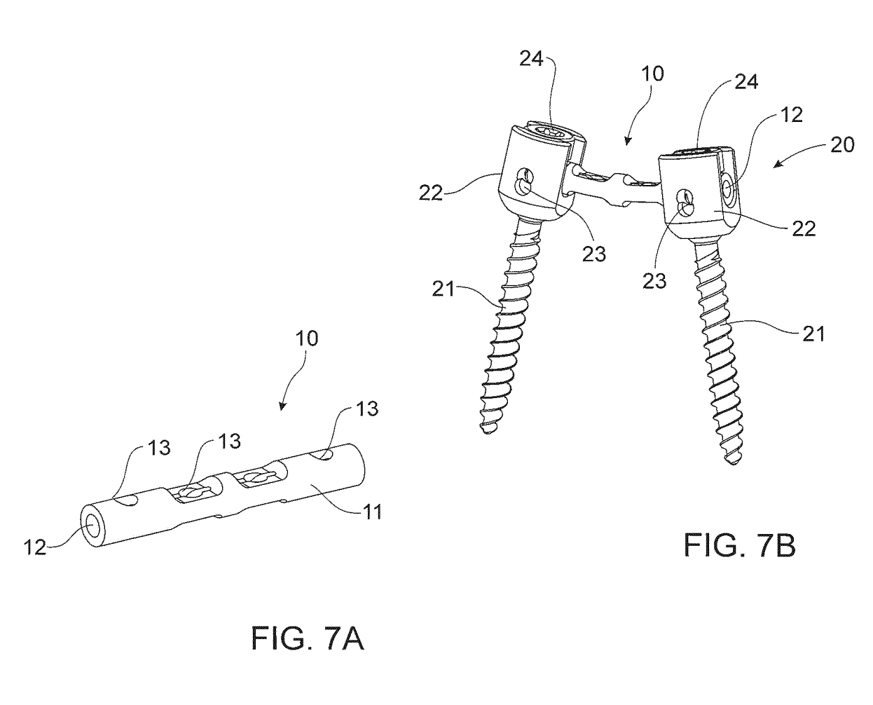

FIG. 7A shows a seventh embodiment of a spinal rod 10. In this embodiment, there are four rod fenestrations 13 that extend through the longitudinal member 11. Again, a single hollow 12 also extends through the longitudinal member 11. The longitudinal member 11 is narrower in some portions than other to simply bending of the longitudinal member 11.

FIG. 7B shows the spinal rod 10 shown in FIG. 7A attached to two bone screws 20. The bone screws 20 shown in FIG. 7A are the same as the bone screws 20 shown in FIG. 6B.

In use, bone screws 20 are located within spinal vertebrae using open or minimally invasive surgical techniques. A degree of stripping of the adjacent transverse processes is then conducted using a long handle elevator.

A variety of different bone screws 20 that have or may not have screw head fenestrations 23 in lateral wall of screw head 22 may be used. Lateral screw head fenestration/s may be used if graft material flow is required down and laterally onto adjacent transverse process. Medial screw head fenestration/s can be used if there is a facet joint milled bony facet defect or a facet cage that requires filling.

The facet joint may be milled medially, if required, and a facet cage inserted as necessary. Decompression of neural structures is also performed if necessary. A transforaminal lumbar interbody fusion (TLIF) and/or posterior lumbar interbody fusion (PLIF) is performed if required.

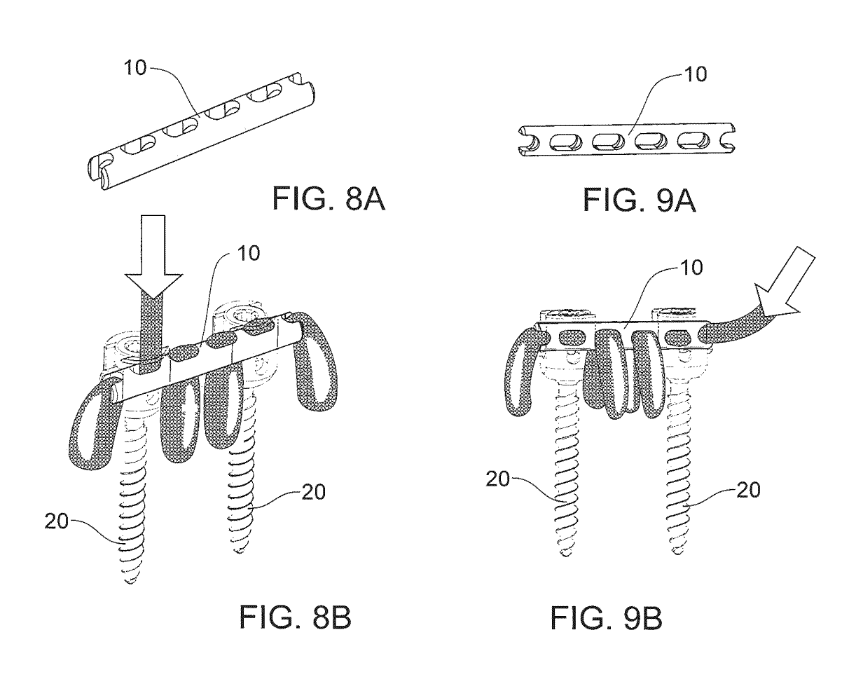

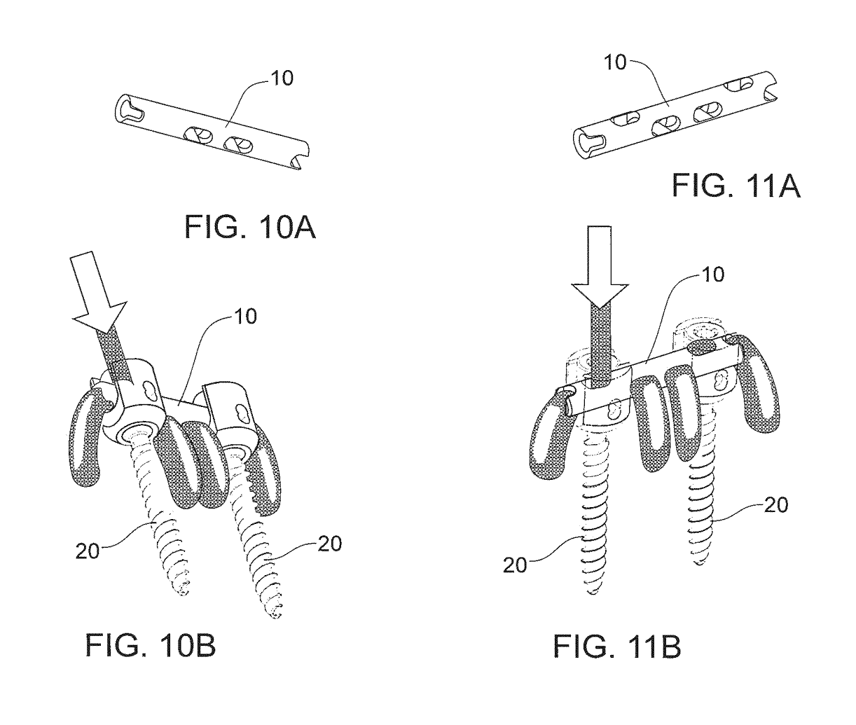

A spinal rod 10 may be selected based on its appropriate length, number of rod fenestrations 13 and position of rod fenestrations 13. The spinal rod 10 is then located between screw head(s) 22 on each side of the spine. The spinal rod 10 is rotated to a desired position in order to locate the rod fenestrations 13 in the most advantageous position. For example, a rod fenestration 13 may be positioned adjacent a screw head fenestration 23. Locking caps 24 are then used to fasten the spinal rod 10 to the bone screw 20. FIGS. 8A to 11A show different spinal rods 10 whilst FIGS. 8B to 11B show the different spinal rods 10 connected to bone screws 20 with graft material flowing from the spinal rods 20.

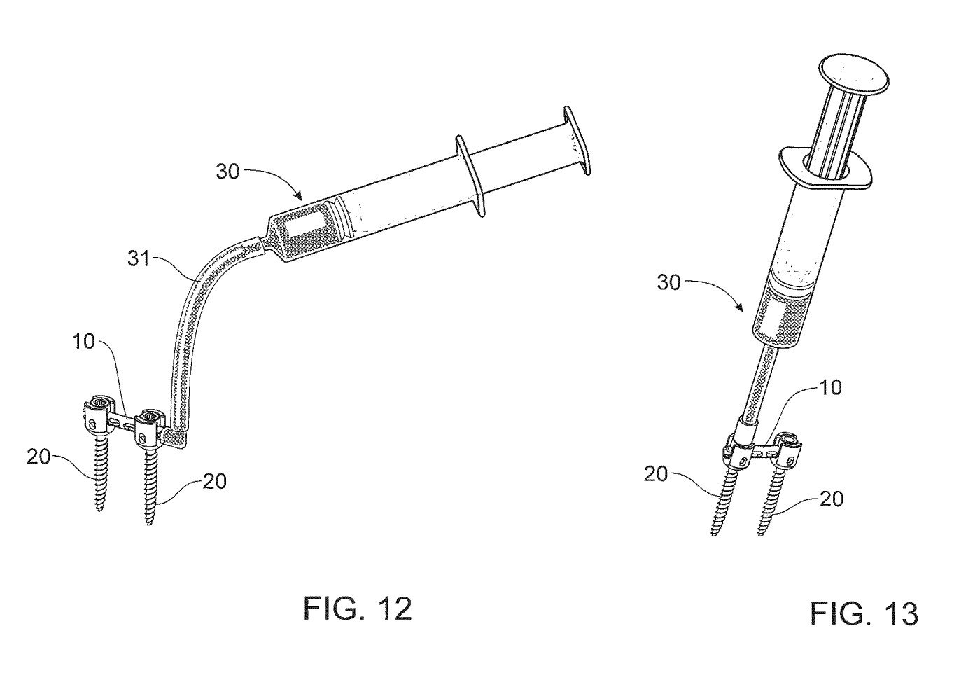

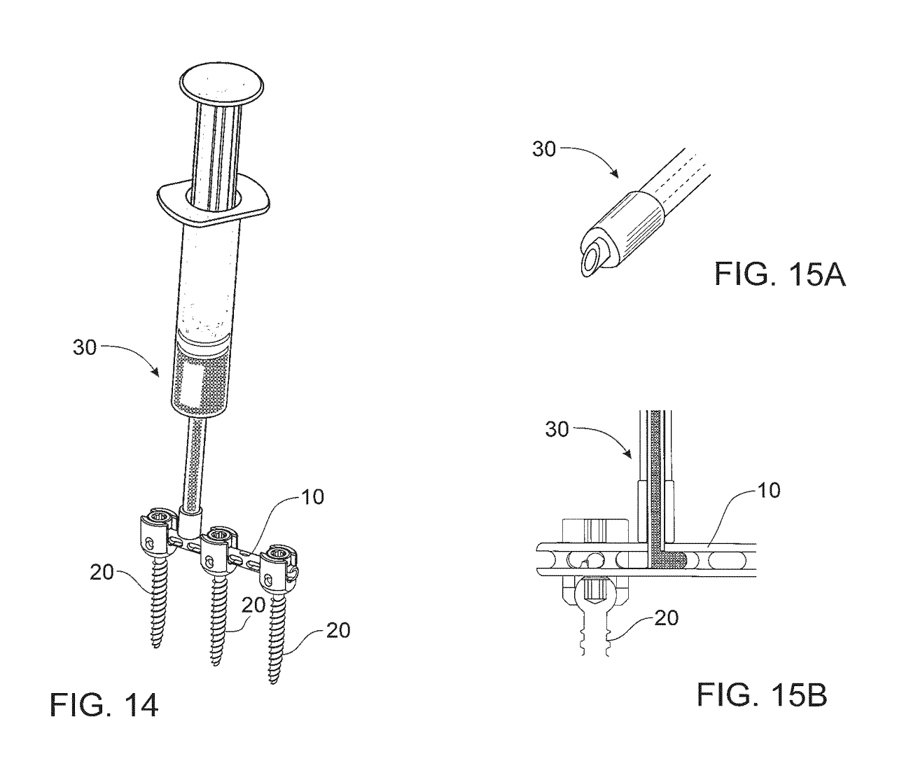

A delivery tool 30 (for MIS particularly), is fluidly coupled to the hollow. The delivery tool 30 may be directly attached to the hollow 12 using a pipe 31 (or hose or nozzle) as shown in FIG. 12 or accessed through the locking cap 24 shown in FIG. 13 or accessed through a rod fenestration 13 as shown in FIG. 14. The delivery tool 30 is used to deliver graft material into the hollow 12 and may be shaped to deliver graft material in different directions as shown in FIGS. 15A and 15B. Alternately, a bone morphogenic protein sponge graft or autograft can be placed in the hollow 12 before insertion to the screw heads 22.

The delivery tool 30 is then used to inject bone graft material to each side (i.e. right and left). The position of graft material can then be determined with fluoroscopy (if graft material is radio-opaque). Additional graft material can be added as is required. The surgical technique is then completed by closing the wound of patient in the usual fashion.

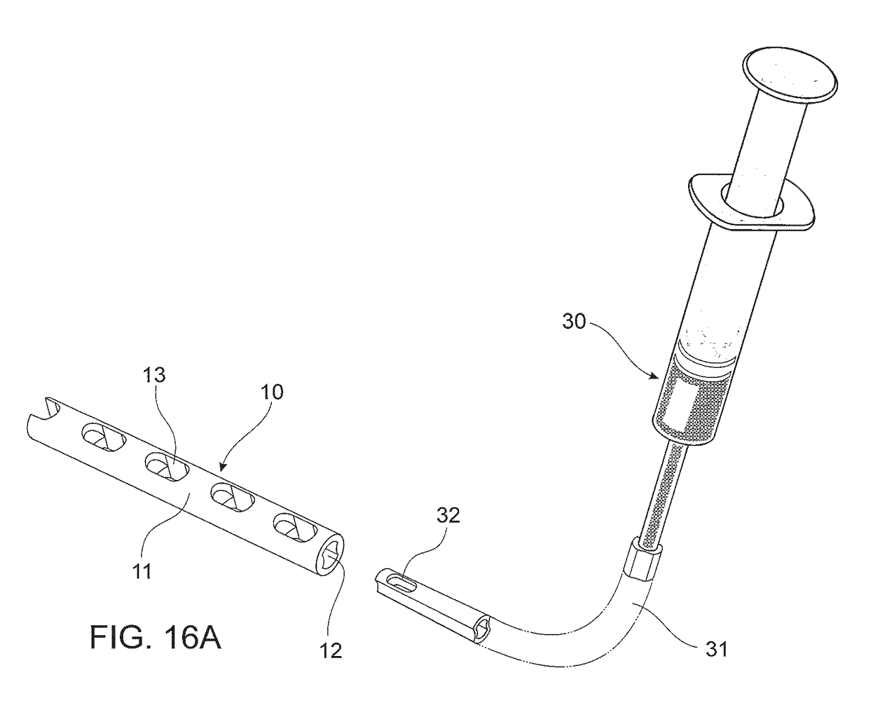

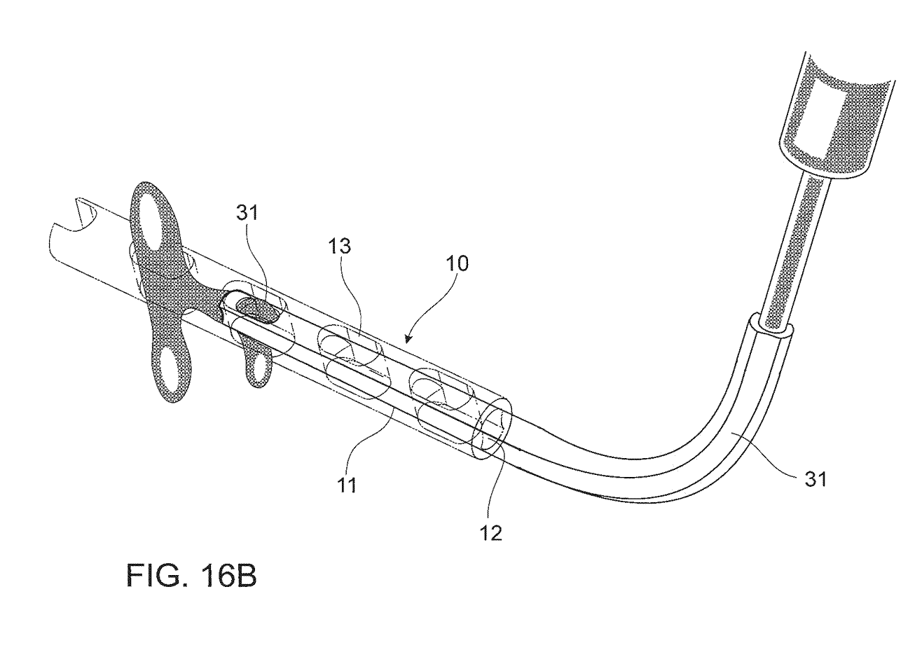

It should be appreciated that the delivery tool 30 can be modified, shown in FIGS. 16A and 16B, to provide a more controlled release of graft material into an associated spinal rod 10. In this embodiment, the delivery tool 30 includes an elongate pipe 31 which is non-cylindrical. Further, outlet holes 32 are located adjacent the end of the pipe 31 as opposed to at the end of the pipe 31. The associated spinal rod 10 includes hollow 12 which is also non-cylindrical and substantially matches the transverse cross section of the pipe 31.

In use, the pipe 31 is inserted into the hollow 12 passed the last fenestration 13. As the pipe 31 is withdrawn from the hollow, graft material is fed through the pipe 31 and through specific fenestrations 13. Due to the matching transverse cross sectional shape of the pipe 31 and the hollow 12, the outlet holes 32 of the pipe align with the fenestrations 13 as the pipe 31 is withdrawn from the hollow 12. This arrangement enables a controlled release of graft material that would otherwise not be possible.

Essentially, the surgical technique above surrounds an attempt to achieve a posterior spinal fusion anywhere from the occiput level (base of skull) to sacrum or pelvic level. The hollow spinal rod can be used at any level in the spine as listed above and that can be used in conjunction with a bone screw or lateral mass screw.

The surgical technique uses a hollow spinal rod that has rod fenestrations to permit the passage of bone graft material (most likely injectable bone graft forms), into the spinal rod when in situ and thus allow flow of graft material onto adjacent bone surfaces such as the transverse processes, the lamina and the facet joints. Similarly, the screw head fenestrations in the screw head also allow the flow of graft material onto adjacent bone surfaces such as the transverse processes, the lamina and the facet joints.

It should be appreciated that various other changes and modifications may be made to the embodiments described without departing from the spirit or scope of the invention.

1. A spinal rod able to be used with any bone fixation device, the spinal rod comprising:
a longitudinal member having at least one hollow extending therethrough.

2. The spinal rod as claimed in claim 1 wherein the longitudinal member is made from a combination of segments.

3. The spinal rod as claimed in claim 1 wherein the hollow extends completely through the longitudinal member.

4. The spinal rod as claimed in claim 1 wherein the hollow extends partially through the longitudinal member.

5. The spinal rod as claimed in claim 1 wherein the spinal rod comprises at least one rod fenestration that extends through the longitudinal member.

6. The spinal rod of claim 1 wherein the longitudinal member is made from at least one material that allow bone growth on the surface of the longitudinal member.

7. The spinal rod of claim 6 wherein the material used is selected from the group comprising titanium alloy, CrCo, 316L stainless steel, PEEK™ Nitinol or ceramic.

8. The spinal rod of claim 1 wherein the longitudinal member is straight.

9. The spinal rod of claim 1 wherein the longitudinal member is pre-bent.

10. The spinal rod of claim 1 wherein the longitudinal member includes at least one ridged beam.

11. The spinal rod of claim 1 wherein the longitudinal member includes at least one strengthening rib.

12. The spinal rod of claim 1 wherein internal dimensions of the hollow member are uniform.

13. The spinal rod of claim 1 wherein internal dimensions of the hollow member are non-uniform.

14. The spinal rod of claim 1 wherein the hollow of the longitudinal member is pre-packed with graft material.

15. The spinal rod of claim 1 wherein the hollow is open at both ends.

16. The spinal rod of claim 1 wherein the hollow is open at one ends.

17. A spinal fusion system comprising:
at least one spinal rod comprising a longitudinal member having at least one hollow extending through the longitudinal member; and at least one rod fenestration that extends through the longitudinal member; and
at least two bone screws for attachment to a spine, each bone screw comprising a threaded shaft for location within the spine, a head attached to the threaded shaft, the head able to be used for attachment to a rod, the head including at least one fenestration;
wherein at least one spinal rod is attached to the respective heads to the bone screws.

18. The spinal fusion system of claim 17 wherein the rod fenestrations are aligned with screw head fenestrations to allow graft material to pass from the longitudinal member and through the screw head.

19. A method of surgery including the steps of:
attaching a spinal rod to at least one bone screw, the spinal rod comprising a longitudinal member having at least one hollow extending through the longitudinal member; and at least one rod fenestrations that extends through the longitudinal member; and
injecting bone graft into the hollow of the longitudinal member.

20. The method of claim 19 further including the steps of:
setting the bone screw in a spine, each bone screw comprising a threaded shaft for location within the spine, a head attached to the threaded shaft, the head able to be used for attachment to a rod, the head including at least one fenestration; wherein the shaft is rotated so that the fenestration in the head is in a desired location;
rotating the rod with respect to the bone screw such that a rod fenestration is located adjacent a screw head fenestration.
